SEQUENCE LISTING (7 sequences)

SEQ ID NO: 1 - protein, 418 aa, Homo sapiens
MRSLLLLSAFCLLEAALAAEVKKPAAAAAPGTAEKLSPKAATLAERSAGLAFSLYQAMAKDQAVENILVSPVVVASSLGLVSLGGKATTASQAKAVLSAEQLRDEEVHAGLGELLRSLSNSTARNVTWKLGSRLYGPSSVSFADDFVRSSKQHYNCEHSKINFRDKRSALQSINEWAAQTTDGKLPEVTKDVERTDGALLVNAMFFKPHWDEKFHHKMVDNRGFMVTRSYTVGVMMMHRTGLYNYYDDEKEKLQIVEMPLAHKLSSLIILMPHHVEPLERLEKLLTKEQLKIWMGKMQKKAVAISLPKGVVEVTHDLQKHLAGLGLTEAIDKNKADLSRMSGKKDLYLASVFHATAFELDTDGNPFDQDIYGREELRSPKLFYADHPFIFLVRDTQSGSLLFIGRLVRPKGDKMRDEL

SEQ ID NO: 2 - protein, 413 aa, Homo sapiens
  MOD_RES (277): Variable amino acid
  MOD_RES (310): Variable amino acid
SLLLLSAFCLLAVALATEVKKPAATAAPGTAEKLSPKAATLAEHSAGLAFSLYQAMAKDQAVENILVSPVVVASSLGLVSLGGKATTASEAKAVLSAKQLSDEEVHAGVGEPLRSLSNSTARNVTWKLCSRLGPSSVSFADDFVRSSKQHYNCEHSKINFHDKRSALQSIHEWAVQTTDGKLPKVTKDMECMDGALLVNTMFFKPHWNEKFHHKMVENRGFMVTRFYTVGVMVMHQTGLYNYYDNEKEKLQIVEMPLAHKLSSLIILMPHHVEPLEXLKKLVTKEQLKIWMGKKQKPVAISLPKRVVEVXHDLQKLLARLGLTEAIDKNKANLSRMPHKKDLYLTSVFHATAFELDTDGNSFDQDIYGSKELRSPKLFYSDHPFIFLVWDTQSGSLLFTGHLVRPKVDKMQDE

SEQ ID NO: 3 - protein, 18 aa, Homo sapiens
LCSRLGPSSVSFADDFVR

SEQ ID NO: 4 - protein, 15 aa, Homo sapiens
HLAGLGLTEAIDKNK

SEQ ID NO: 5 - protein, 13 aa, Homo sapiens
LFYADHPFIFLVR

SEQ ID NO: 6 - protein, 17 aa, Homo sapiens
LSSLIILMPHHVEPLER

SEQ ID NO: 7 - protein, 23 aa, Homo sapiens
SALQSINEWAAQTTDGKLPEVTK

(19) United States
(12) Patent Application Publication
(10) Pub. No.: US 2007/0161062 A1
(43) Pub. Date: Jul. 12, 2007

(54) Protein CBP2 as a marker for colorectal cancer

(76) Inventors: Michael Tacke, Muenchen (DE); Marie-Luise Hagmann, Penzberg (DE); Johann Karl, Peissenberg (DE); Stefan Palme, Penzberg (DE); Michael Pfeffer, Penzberg (DE); Wolfgang Rollinger, Polling (DE); Michael Thierolf, Penzberg (DE); Werner Zolg, Weilheim-Unterhausen (DE)

Correspondence Address: ROCHE DIAGNOSTICS OPERATIONS INC., 9115 Hague Road, Indianapolis, IN, 46250-0457, US

(21) Appl. No.: 11/640,511

(22) Filed: Dec. 15, 2006

Related U.S. Application Data
(63) Continuation of application No. PCT/EP05/06525, filed on Jun. 17, 2005.

(30) Foreign Application Priority Data
Jun. 18, 2004 (EP) 04014310.9

(51) Int. Cl. G01N 33/574

(52) U.S. Cl. 435/7.23

(57) ABSTRACT

The present invention relates to the diagnosis of colorectal cancer. It discloses the use of protein CBP2 (collagen-binding protein 2) in the diagnosis of colorectal cancer. It relates to a method for diagnosis of colorectal cancer from a liquid sample, derived from an individual by measuring CBP2 in said sample. Measurement of CBP2 can, e.g., be used in the early detection or diagnosis of colorectal cancer.

### RELATED APPLICATIONS

This application is a continuation of PCT/EP2005/006525 filed Jun. 17, 2005 and claims priority to EP 04014310.9 filed Jun. 18, 2004.

### FIELD OF THE INVENTION

The present invention relates to the diagnosis of colorectal cancer. It discloses the use of CBP2 (collagen-binding protein 2) in the diagnosis of colorectal cancer. Furthermore, it especially relates to a method for diagnosis of colorectal cancer from a liquid sample, derived from an individual by measuring CBP2 in said sample. Measurement of CBP2 can, e.g., be used in the early detection of colorectal cancer or in the surveillance of patients who undergo surgery.

### BACKGROUND OF THE INVENTION

Cancer remains a major public health challenge despite progress in detection and therapy. Amongst the various types of cancer, colorectal cancer (CRC) is one of the most frequent cancers in the Western world.

Colorectal cancer most frequently progresses from adenomas (polyps) to malignant carcinomas. The different stages of CRC used to be classified according to Dukes' stages A to D.

The staging of cancer is the classification of the disease in terms of extent, progression, and severity. It groups cancer patients so that generalizations can be made about prognosis and the choice of therapy.

Today, the TNM system is the most widely used classification of the anatomical extent of cancer. It represents an internationally accepted, uniform staging system. There are three basic variables: T (the extent of the primary tumor), N (the status of regional lymph nodes) and M (the presence or absence of distant metastases). The TNM criteria are published by the UICC (International Union Against Cancer), edition, 1997 (Sobin, L. H., and Fleming, I. D., TNM 80 (1997) 1803-4)

What is especially important is that early diagnosis of CRC translates to a much better prognosis. Malignant tumors of the colorectum arise from benign tumors, i.e. from adenoma. Therefore, best prognosis have those patients diagnosed at the adenoma stage. Patients diagnosed as early as in stage Tᵢₛ, N0, M0 or T1-3; N0; M0, if treated properly have a more than 90% chance of survival 5 years after diagnosis as compared to a 5-years survival rate of only 10% for patients diagnosed when distant metastases are already present.

In the sense of the present invention early diagnosis of CRC refers to a diagnosis at a pre-malignant state (adenoma) or at a tumor stage where no metastases at all (neither proximal nor distal), i.e., adenoma, Tᵢₛ, N0, M0 or T1-4; N0; M0 are present. Tᵢₛ denotes carcinoma in situ.

It is further preferred, that CRC is diagnosed when it has not yet fully grown through the bowel wall and thus neither the visceral peritoneum is perforated nor other organs or structures are invaded, i.e., that diagnosis is made at stage Tᵢₛ, N0, M0 or T1-3; N0; M0 (Tᵢₛ-3; N0; M0).

The earlier cancer can be detected/diagnosed, the better is the overall survival rate. This is especially true for CRC. The prognosis in advanced stages of tumor is poor. More than one third of the patients will die from progressive disease within five years after diagnosis, corresponding to a survival rate of about 40% for five years. Current treatment is only curing a fraction of the patients and clearly has the best effect on those patients diagnosed in an early stage of disease.

With regard to CRC as a public health problem, it is essential that more effective screening and preventative measures for colorectal cancer be developed.

The earliest detection procedures available at present for colorectal cancer involve using tests for fecal blood or endoscopic procedures. However, significant tumor size must typically exist before fecal blood is detected. The sensitivity of the guaiac-based fecal occult blood tests is ˜26%, which means 74% of patients with malignant lesions will remain undetected (Ahlquist, D. A., Gastroenterol. Clin. North Am. 26 (1997) 41-55). The visualization of precancerous and cancerous lesions represents the best approach to early detection, but colonoscopy is invasive with significant costs, risks, and complications (Silvis, S. E., et al., JAMA 235 (1976) 928-930; Geenen, J. E., et al., Am. J. Dig. Dis. 20 (1975) 231-235; Anderson, W. F., et al., J. Natl. Cancer Institute 94 (2002) 1126-1133).

In order to be of clinical utility a new diagnostic marker as a single marker should be at least as good as the best single marker known in the art. Or, a new marker should lead to a progress in diagnostic sensitivity and/or specificity either if used alone or in combination with one or more other markers, respectively. The diagnostic sensitivity and/or specificity of a test is best assessed by its receiver-operating characteristics, which will be described in detail below.

The clinical utility of biochemical markers in colorectal cancer has recently been reviewed by the European Group on Tumor Markers (EGTM) (Duffy, M. J., et al Europ. J. of Cancer 39 (2003) 718-727).

At present, primarily diagnostic blood tests based on the detection of carcinoembryonic antigen (CEA), a tumor-associated glycoprotein, are available to assist diagnosis in the field of CRC. CEA is increased in 95% of tissue samples obtained from patients with colorectal, gastric, and pancreatic cancers and in the majority of breast, lung, and head and neck carcinomas (Goldenberg, D. M., et al., J. Natl. Cancer Inst. (Bethesda) 57 (1976) 11-22). Elevated CEA levels have also been reported in patients with nonmalignant disease, and many patients with colorectal cancer have normal CEA levels in the serum, especially during the early stage of the disease (Carriquiry, L. A., and Pineyro, A., Dis. Colon Rectum 42 (1999) 921-929; Herrera, M. A., et al., Ann. Surg. 183 (1976) 5-9; Wanebo, H. J., et al., N. Engl. J. Med. 299 (1978) 448-451). The utility of CEA as measured from serum or plasma in detecting recurrences is reportedly controversial and has yet to be widely applied (Martell, R. E., et al., Int. J. Biol. Markers 13 (1998) 145-149; Moertel, C. G., et al., JAMA 270 (1993) 943-947).

In light of the available data, serum CEA determination possesses neither the sensitivity nor the specificity to enable its use as a screening test for colorectal cancer in the asymptomatic population (Reynoso, G., et al., JAMA 220 (1972) 361-365; Sturgeon, C., Clinical Chemistry 48 (2002) 1151-1159).

Whole blood, serum or plasma are the most widely used sources of sample in clinical routine. The identification of an early CRC tumor marker that would aid in the reliable cancer detection or provide early prognostic information could lead to a diagnostic assay that would greatly aid in the diagnosis and in the management of this disease. Therefore, an urgent clinical need exists to improve the in vitro assessment of CRC. It is especially important to improve the early diagnosis of CRC, since for patients diagnosed early on chances of survival are much higher as compared to those diagnosed at a progressed stage of disease.

### SUMMARY OF THE INVENTION

It was the task of the present invention to investigate whether a biochemical marker can be identified which may be used in assessing CRC.

Surprisingly, it has been found that use of the marker CBP2 can at least partially overcome the problems known from the state of the art.

The present invention therefore relates to a method for assessing colorectal cancer in vitro by biochemical markers comprising measuring in a sample the concentration of a) CBP2, and b) using the concentration determined in step (a) in the assessment of colorectal cancer.

Another preferred embodiment of the invention is a method for assessing colorectal cancer comprising the steps of a) contacting a liquid sample obtained from an individual with a specific binding agent for CBP2 under conditions appropriate for formation of a complex between said binding agent and CBP2, and b) correlating the amount of complex formed in (a) to the assessment of colorectal cancer.

Yet another preferred embodiment of the invention relates to a method for assessing colorectal cancer in vitro by biochemical markers, comprising measuring in a sample the concentration of CBP2 and of one or more other marker of colorectal cancer and using the concentrations determined in the assessment of colorectal cancer.

The present invention also relates to the use of a marker panel comprising at least CBP2 and CYFRA 21-1 in the assessment of CRC.

The present invention also relates to the use of a marker panel comprising at least CBP2 and NSE in the assessment of CRC.

The present invention also relates to the use of a marker panel comprising at least CBP2 and CEA in the assessment of CRC.

The present invention also relates to the use of a marker panel comprising at least CBP2 and NNMT in the assessment of CRC.

The present invention also relates to the use of a marker panel comprising at least CBP2 and CA 19-9 in the assessment of CRC.

The present invention also relates to the use of a marker panel comprising at least CBP2 and CA 72-4 in the assessment of CRC.

The present invention also provides a kit for performing the method according to the present invention comprising at least the reagents required to specifically measure CBP2 and CYFRA 21-1, respectively, and optionally auxiliary reagents for performing the measurement.

The present invention also provides a kit for performing the method according to the present invention comprising at least the reagents required to specifically measure CBP2 and NSE, respectively, and optionally auxiliary reagents for performing the measurement.

In a further preferred embodiment the present invention relates to a method for assessing colorectal cancer in vitro comprising measuring in a sample the concentration of a) CBP2, b) optionally one or more other marker of colorectal cancer, and c) using the concentrations determined in step (a) and optionally step (b) in the assessment of colorectal cancer.

### DETAILED DESCRIPTION OF THE INVENTION

As used herein, each of the following terms has the meaning associated with it in this section.

The term “marker” or “biochemical marker” as used herein refers to a molecule to be used as a target for analyzing patient test samples. Examples of such molecular targets are proteins or polypeptides themselves as well as antibodies present in a sample. Proteins or polypeptides used as a marker in the present invention are contemplated to include any variants of said protein as well as fragments of said protein or said variant, in particular, immunologically detectable fragments. One of skill in the art would recognize that proteins which are released by cells or present in the extracellular matrix which become damaged, e.g., during inflammation could become degraded or cleaved into such fragments. Certain markers are synthesized in an inactive form, which may be subsequently activated by proteolysis. As the skilled artisan will appreciate, proteins or fragments thereof may also be present as part of a complex. Such complex also may be used as a marker in the sense of the present invention. Variants of a marker polypeptide are encoded by the same gene, but differ in their PI or MW, or both (e.g., as a result of alternative mRNA or pre-mRNA processing, e.g. alternative splicing or limited proteolysis) and in addition, or in the alternative, may arise from differential post-translational modification (e.g., glycosylation, acylation, and/or phosphorylation).

The term “assessing colorectal cancer” is used to indicate that the method according to the present invention will (alone or together with other markers or variables, e.g., the criteria set forth by the UICC (UICC (International Union Against Cancer), Sobin, L. H., Wittekind, Ch. (eds), TNM Classification of Malignant Tumours, fifth edition, 1997)) e.g., aid the physician to establish or confirm the absence or presence of CRC or aid the physician in the prognosis, the detection of recurrence (follow-up of patients after surgery) and/or the monitoring of treatment, especially of chemotherapy.

The term “sample” as used herein refers to a biological sample obtained for the purpose of evaluation in vitro. In the methods of the present invention, the sample or patient sample preferably may comprise any body fluid. Preferred test samples include blood, serum, plasma, urine, saliva, and synovial fluid. Preferred samples are whole blood, serum, plasma or synovial fluid, with plasma or serum being most preferred. As the skilled artisan will appreciate, any such assessment is made in vitro. The patient sample is discarded afterwards. The patient sample is solely used for the in vitro method of the invention and the material of the patient sample is not transferred back into the patient's body. Typically, the sample is a liquid sample, e.g., whole blood, serum, or plasma.

In a preferred embodiment the present invention relates to a method for assessing CRC in vitro by biochemical markers, comprising measuring in a sample the concentration of CBP2 and using the concentration determined in the assessment of CRC.

The protein CBP2 (collagen-binding protein 2; serine [or cysteine] proteinase inhibitor, clade H, member 1 precursor; colligin-2; heat shock protein 47; rheumatoid arthritis antigen A-4747; RA A47; SerpinH2; gp46; heat shock protein 47; rheumatoid arthritis antigen A 47) is characterized by the sequence given SEQ ID No.1 and SEQ ID No. 2 or an isoform thereof.

Collagen-binding proteins, or colligins, are glycoproteins that bind specifically to collagen type I, collagen type IV and gelatin. Colligins are characterized by an amino acid structure that includes an N-terminal hydrophobic signal sequence and 2 putative N-linked oligosaccharide attachment sites (Clarke, E. P., et al., J. Biol. Chem. 266 (1991) 17230-17235).

Colligins also have a C-terminal RDEL sequence that acts as an endoplasmic reticulum (ER) retention sequence. Other features permit the colligin-binding protein of ER to be classified as a serpin (serine-arginine protease inhibitor).

This gene encodes a member of the serpin superfamily of serine proteinase inhibitors. Its expression is induced by heat shock. The protein localizes to the endoplasmic reticulum lumen and binds collagen; thus it is thought to be a molecular chaperone involved in the maturation of collagen molecules. Autoantibodies to this protein have been found in patients with rheumatoid arthritis.

CBP2 is detected in the cytoplasm of normal squamous epithelial cells, neither serpin is detected normally in the serum. Thus, their presence in the circulation at relatively high concentrations suggests that malignant epithelial cells are re-directing serpin activity to the fluid phase via an active secretory process. Using subcellular fractionation, CBP2 was found exclusively in the cytosol and were not associated with nuclei, mitochondria, lysosomes, microtubules, actin or the Golgi. In contrast to previous reports, metabolic labeling and pulse-chase experiments showed that neither non-stimulated nor TNFalpha/PMA-stimulated squamous carcinoma cells appreciably secreted these ov-serpins into the medium. Collectively, these data suggest that the major site of serpin inhibitory activity remains within the cytosol and that their presence in the sera of patients with advanced squamous-cell carcinomas may be due to their passive release into the circulation (Uemura, Y., et al., Int. J. Cancer 89 (2000) 368-77).

As obvious to the skilled artisan, the present invention shall not be construed to be limited to the full-length protein CBP2 of SEQ ID NO: 1 or SEQ ID NO:2. Physiological or artificial fragments of CBP2, secondary modifications of CBP2, as well as allelic variants of CBP2 are also encompassed by the present invention. Artificial fragments preferably encompass a peptide produced synthetically or by recombinant techniques, which at least comprises one epitope of diagnostic interest consisting of at least 6 contiguous amino acids as derived from the sequence disclosed in SEQ ID NO: 1 or SEQ ID NO:2. Such fragment may advantageously be used for generation of antibodies or as a standard in an immunoassay. More preferred the artificial fragment comprises at least two epitopes of interest appropriate for setting up a sandwich immunoassay. Preferably, full-length CBP2 or a physiological variant of this marker is detected in a method according to the present invention,

The assessment method according to the present invention is based on a liquid sample which is derived from an individual. Unlike to methods known from the art CBP2 is specifically measured from this liquid sample by use of a specific binding agent.

A specific binding agent is, e.g., a receptor for CBP2, a lectin binding to CBP2 or an antibody to CBP2. A specific binding agent has at least an affinity of 10⁷ l/mol for its corresponding target molecule. The specific binding agent preferably has an affinity of 10⁸ l/mol or even more preferred of 10⁹ l/mol for its target molecule. As the skilled artisan will appreciate the term specific is used to indicate that other biomolecules present in the sample do not significantly bind to the binding agent specific for CBP2. Preferably, the level of binding to a biomolecule other than the target molecule results in a binding affinity which is only 10%, more preferably only 5% of the affinity of the target molecule or less. A most preferred specific binding agent will fulfill both the above minimum criteria for affinity as well as for specificity.

A specific binding agent preferably is an antibody binding to CBP2. The term antibody refers to a polyclonal antibody, a monoclonal antibody, fragments of such antibodies, as well as to genetic constructs comprising the binding domain of an antibody.

Any antibody fragment retaining the above criteria of a specific binding agent can be used. Antibodies are generated by state of the art procedures, e.g., as described in Tijssen (Tijssen, P., Practice and theory of enzyme immunoassays 11 (1990) the whole book, especially pages 43-78; Elsevier, Amsterdam). In addition, the skilled artisan is well aware of methods based on immunosorbents that can be used for the specific isolation of antibodies. By these means the quality of polyclonal antibodies and hence their performance in immunoassays can be enhanced. (Tijssen, P., supra, pages 108-115).

For the achievements as disclosed in the present invention polyclonal antibodies raised in rabbits have been used. However, clearly also polyclonal antibodies from different species , e.g. rats or guinea pigs, as well as monoclonal antibodies can also be used. Since monoclonal antibodies can be produced in any amount required with constant properties, they represent ideal tools in development of an assay for clinical routine. The generation and use of monoclonal antibodies to CBP2 in a method according to the present invention is yet another preferred embodiment.

As the skilled artisan will appreciate now, that CBP2 has been identified as a marker which is useful in the diagnosis of CRC, alternative ways may be used to reach a result comparable to the achievements of the present invention. For example, alternative strategies to generate antibodies may be used. Such strategies comprise amongst others the use of synthetic peptides, representing an epitope of CBP2 for immunization. Alternatively, DNA Immunization also known as DNA vaccination may be used.

For measurement the liquid sample obtained from an individual is incubated with the specific binding agent for CBP2 under conditions appropriate for formation of a binding agent CBP2-complex. Such conditions need not be specified, since the skilled artisan without any inventive effort can easily identify such appropriate incubation conditions.

As a final step according to the method disclosed in the present invention the amount of complex is measured and correlated to the diagnosis of CRC. As the skilled artisan will appreciate there are numerous methods to measure the amount of the specific binding agent CBP2-complex all described in detail in relevant textbooks (cf., e.g., Tijssen P., supra, or Diamandis, et al., eds. (1996) Immunoassay, Academic Press, Boston).

Preferably CBP2 is detected in a sandwich type assay format. In such assay a first specific binding agent is used to capture CBP2 on the one side and a second specific binding agent, which is labeled to be directly or indirectly detectable is used on the other side.

As mentioned above, it has surprisingly been found that CBP2 can be measured from a liquid sample obtained from an individual sample. No tissue and no biopsy sample is required to apply the marker CBP2 in the assessment of CRC.

In a preferred embodiment the method according to the present invention is practiced with serum as liquid sample material. In a further preferred embodiment the method according to the present invention is practiced with plasma as liquid sample material. In a further preferred embodiment the method according to the present invention is practiced with whole blood as liquid sample material.

Furthermore stool can be prepared in various ways known to the skilled artisan to result in a liquid sample as well. Such sample liquid derived from stool also represents a preferred embodiment according to the present invention.

The inventors of the present invention have surprisingly been able to detect protein CBP2 in a bodily fluid sample. Even more surprising they have been able to demonstrate that the presence of CBP2 in such liquid sample obtained from an individual can be correlated to the assessment of colorectal cancer. Preferably, an antibody to CBP2 is used in a qualitative (CBP2 present or absent) or quantitative (CBP2 amount is determined) immunoassay.

Measuring the level of protein CBP2 has proven very advantageous in the field of CRC. Therefore, in a further preferred embodiment, the present invention relates to use of protein CBP2 as a marker molecule in the assessment of colorectal cancer from a liquid sample obtained from an individual.

The ideal scenario for diagnosis would be a situation wherein a single event or process would cause the respective disease as, e.g., in infectious diseases. In all other cases correct diagnosis can be very difficult, especially when the etiology of the disease is not fully understood as is the case of CRC. As the skilled artisan will appreciate, no biochemical marker, for example in the field of CRC, is diagnostic with 100% specificity and at the same time 100% sensitivity for a given disease. Rather, biochemical markers are used to assess with a certain likelihood or predictive value the presence or absence of a disease. Therefore, in routine clinical diagnosis various clinical symptoms and biological markers are generally considered together in the diagnosis, treatment, and management of the underlying disease.

Biochemical markers can either be determined individually or, in a preferred embodiment of the invention, they can be measured simultaneously using a chip- or a bead-based array technology. The concentrations of the biomarkers are then interpreted independently using an individual cut-off for each marker or they are combined for interpretation.

In a further preferred embodiment of the invention the assessment of colorectal cancer according to the present invention is performed in a method comprising measuring in a sample the concentration of a) CBP2, b) optionally one or more other marker of colorectal cancer, and c) using the concentration determined in step (a) and optionally step (b) in the assessment of colorectal cancer.

Preferably the method for assessment of CRC is performed by measuring the concentration of CBP2 and of one or more other marker and by using the concentration of CBP2 and of the one or more other marker in the assessment of CRC.

The present invention is also directed to a method for assessing CRC in vitro by biochemical markers, comprising measuring in a sample the concentration of CBP2 and of one or more other marker of CRC and using the concentrations determined in the assessment of CRC.

According to the data shown in the Example section the marker CBP2 in the univariate analysis has (at a specificity of about 90%) a sensitivity for CRC of 54.7%. In the assessment of CRC the marker CBP2 will be of advantage in one or more of the following aspects: screening; diagnostic aid; prognosis; monitoring of chemotherapy, and follow-up.

Screening:

CRC is the second most common malignancy of both males and females in developed countries. Because of its high prevalence, its long asymptomatic phase and the presence of premalignant lesions, CRC meets many of the criteria for screening. Clearly, a serum tumor marker which has acceptable sensitivity and specificity would be more suitable for screening than either FOB testing or endoscopy.

As the data given in the Examples section demonstrate CBP2 alone will not suffice to allow for a general screening e.g. of the at risk population for CRC. Most likely no single biochemical marker in the circulation will ever meet the sensitivity and specificity criteria required for screening purposes. Rather it has to be expected that a marker panel will have to be used in CRC screening. The data established in the present invention indicate that the marker CBP2 will form an integral part of a marker panel appropriate for screening purposes. The present invention therefore relates to the use of CBP2 as one marker of a CRC marker panel for CRC screening purposes. The present data further indicate that certain combinations of markers will be advantageous in the screening for CRC. Therefore the present invention also relates to the use of a marker panel comprising CBP2 and CYFRA 21-1, or of a marker panel comprising CBP2 and NSE, or of a marker panel comprising CBP2 and CYFRA 21-1 and NSE for the purpose of screening for CRC.

Diagnostic Aid:

Preoperative CEA values are of limited diagnostic value. Nonetheless the European Committee on Tumor Markers (ECTM) recommends that CFA should be measured before surgery in order to establish a baseline value and for assessing the prognosis. Since CBP2 as a single marker according to the data of the present invention might be at least as good a single marker as CEA or even superior it has to be expected that CBP2 will be used as a diagnostic aid, especially by establishing a baseline value before surgery.

The present invention thus also relates to the use of CBP2 for establishing a baseline value before surgery for CRC.

Prognosis:

The gold standard for determining prognosis in patients with CRC is the extend of disease as defined by the Dukes', TNM or other staging systems, If a marker such as CEA is to be used for predicting outcome, it must: provide stronger prognostic information than that offered by existing staging systems, provide information independent of the existing systems or provide prognostic data within specific subgroups defined by existing criteria, e.g. in Dukes' B or node-negative patients.

Recently, an American Joint Committee on Cancer (AJCC) Consensus Conference suggested that CEA should be added to the TNM staging system for colorectal cancer. The CEA level should be designated as follows: CX, CEA cannot be assessed; CO, CEA not elevated (<5 μg/l) or CEA1, CEA elevated (>5 μg/l) (Compton, C., et al., Cancer 88 (2000) 1739-1757).

As CBP2 alone significantly contributes to the differentiation of CRC patients from healthy controls or from healthy controls plus non-malignant colon diseases, it has to be expected that it will aid in assessing the prognosis of patients suffering from CRC. The level of preoperative CBP2 will most likely be combined with one or more other marker for CRC and/or the TNM staging system, as recommended for CEA by the AJCC. In a preferred embodiment CBP2 is used in the prognosis of patients with CRC.

Monitoring of Chemotherapy:

A number of reports have described the use of CEA in monitoring the treatment of patients with advanced CRC (for review, see Refs. Duffy, M. J., Clin. Hem. 47 (2001) 625-630; Fletcher, R. H., Ann. Int. Med. 104 (1986) 66-73; Anonymous, J. Clin. Oncol. 14 (1996) 2843-2877). Most of these were retrospective, non-randomized and contained small numbers of patients. These studies suggested: a) that patients with a decrease in CEA levels while receiving chemotherapy generally had a better outcome than those patients whose CEA levels failed to decrease and (b) for almost all patients, increases in CEA levels were associated with disease progression.

Due to the data shown in the example section, it has to be expected that CBP2 will be at least as good a marker for monitoring of chemotherapy as CEA. The present invention therefore also relates to the use of CBP2 in the monitoring of CRC patients under chemotherapy.

Follow-Up:

Approximately 50% of patients who undergo surgical resection aimed at cure, later develop recurrent of metastatic disease (Berman, J. M., et al., Lancet 355 (2000) 395-399). Most of these relapses occur within the first 2-3 years of diagnosis and are usually confined to the liver, lungs or locoregional areas. Since recurrent/metastatic disease is invariably fatal, considerable research has focused on its identification at an early and thus potentially treatable stage. Consequently, many of these patients undergo a postoperative surveillance program which frequently includes regular monitoring with CEA.

Serial monitoring with CEA has been shown to detect recurrent/metastatic disease with a sensitivity of approximately of 80%, specificity of approximately 70% and provides an average lead-time of 5 months (for review, see Duffy, M. J., et al. supra and Fletcher, R. H., supra). Furthermore, CEA was the most frequent indicator of recurrence in asymptomatic patients (Pietra, N., et al., Dis. Colon Rectum 41 (1998) 1127-1133 and Graham, R. A., et al., Ann. Surg. 228 (1998) 59-63) and was more cost-effective than radiology for the detection of potentially curable recurrent disease. As regards sites of recurrence/metastasis, CEA was most sensitive (almost 100%) for the detection of liver metastasis. On the other hand, CEA was less reliable for diagnosing locoregional recurrences, the sensitivity being only approximately 60% (Moertel, C. G., et al., Jama 270 (1993) 943-7).

As a compromise between patient convenience, costs and efficiency of disease detection, the EGTM Panel like the CBP2O Panel (Anonymous, J. Clin. Oncol. 14 (1996) 2843-2877) suggests that CEA testing be carried out every 2-3 months for at least 3 years after the initial diagnosis. After 3 years, testing could be carried out less frequently, e.g. every 6 months. No evidence exists, however, to support this frequency of testing.

As the above discussion of the state of the art shows, that the follow-up of patients with CRC after surgery is one of the most important fields of use for an appropriate biochemical marker. Due to the high sensitivity of CBP2 in the CRC patients investigated it is expected that CBP2 alone or in combination with one or more other marker will be of great help in the follow-up of CRC patients, especially in CRC patients after surgery. The use of a marker panel comprising CBP2 and one or more other marker of CRC in the follow-up of CRC patients represents a further preferred embodiment of the present invention.

The present invention discloses and therefore in a preferred embodiment relates to the use of CBP2 in the diagnostic field of CRC or in the assessment of CRC, respectively.

In yet a further preferred embodiment the present invention relates to the use of CBP2 as a marker molecule for colorectal cancer in combination with one or more marker molecules for colorectal cancer in the assessment of colorectal cancer from a liquid sample obtained from an individual. In this regard, the expression “one or more” denotes 1 to 20, preferably 1 to 10, preferably 1 to 5, more preferred 3 or 4. CBP2 and the one or more other marker form a CRC marker panel.

Thus, a preferred embodiment of the present invention is the use of CBP2 as a marker molecule for colorectal cancer in combination with one or more marker molecules for colorectal cancer in the assessment of colorectal cancer from a liquid sample obtained from an individual. Preferred selected other CRC markers with which the measurement of CBP2 may be combined are NSE, CYFRA 21-1, NMMT, CA 19-9, CA 72-4, and/or CEA. Yet further preferred the marker panel used in the assessment of CRC comprises CBP2 and at least one other marker molecule selected from the group consisting of NSE, CYFRA 21-1 and NMMT.

The markers which preferably are combined with CBP2 or which form part of the CRC marker panel comprising CBP2, respectively, are discussed in more detail below.

NSE (Neuron-Specific Enolase)

The glycolytic enzyme enolase (2-phospho-D-glycerate hydrolase, EC 4.2.1.11, molecular weight approx. 80 kD) occurs in a variety of dimeric isoforms comprising three immunologically different subunits termed α, β, and γ. The α-subunit of enolase occurs in numerous types of tissue in mammals, whereas the β-subunitis found mainly in the heart and in striated musculature. The enolase isoforms αγ and γγ, which are referred to as neuron-specific enolase (NSE) or γ-enolase, are primarily detectable in high concentrations in neurons and neuro-endocrine cells as well as in tumors originating from them. (Lamerz, R., NSE (Neuronen-spezifische Enolase), γ-Enolase. In: Thomas L (ed) Clinical Laboratory Diagnosis, TH-Books, Frankfurt, 1^{st} English Edition 1998: 979-981, 5. deutsche Auflage 1998:1000-1003)

NSE is described as the marker of first choice in the monitoring of small cell bronchial carcinoma, (Lamerz, R., supra), whereas CYFRA 21-1 is superior to NSE for non-small cell bronchial carcinoma. (Ebert, W., et al., Eur. J. Clin. Chem. Clin. Biochem 32 (1994) 189-199).

Elevated NSE concentrations are found in 60-81% of cases of small cell bronchial carcinoma.

For NSE there is no correlation to the site of metastasis or to cerebal metastasis, but there is good correlation to the clinical stage, i.e. the extent of the disease.

In response to chemotherapy there is a temporary rise in the NSE level 24-72 hours after the first therapy cycle as a result of cytolysis of the tumor cells. This is followed within a week or by the end of the first therapy cycle by a rapid fall in the serum values (which were elevated prior to therapy). By contrast, non-responders to therapy display levels which are constantly elevated or fail to fall into the reference range. During remission, 80-96% of the patients have normal values. Rising NSE values are found in cases of relapse. The rise occurs in some cases with a latent period of 1-4 months, is often exponential (with a doubling time of 10-94 days) and correlates with the survival period. NSE is useful as a single prognostic factor and activity marker during the monitoring of therapy and the course of the disease in small cell bronchial carcinoma: diagnostic sensitivity 93%, positive predictive value 92% (Lamerz R., supra)

In neuroblastoma NSE serum values above 30 ng/ml are found in 62% of the affected children. The medians rise in accordance with the stage of the disease. There is a significant correlation between the magnitude or frequency of pathological NSE values and the stage of disease; there is an inverse correlation with illness-free survival.

68-73% of the patients with seminoma have a clinically significant NSE elevation. (Lamerz, R., supra). There is a utilizable correlation with the clinical course of the disease.

NSE has also been measured in other tumors: Non-pulmonary malignant diseases show values above 25 ng/ml in 22% of the cases (carcinomas in all stages). Brain tumors such as glioma, miningioma, neurofibroma, and neurinoma are only occasionally accompanied by elevated serum NSE values. In primary brain tumors or brain metastasis and in malignant melanoma and phaeochromocytoma, elevated NSE-values can occur in the CSF (cerebrospinal fluid). Increased NSE concentrations have been reported for 14% of organ-restricted and 46% of metastasizing renal carcinomas, with a correlation to the grade as an independent prognosis factor.

In benign disease elevated serum NSE concentrations (>12 ng/ml) have been found in patients with benign pulmonary diseases and cerebral diseases. Elevated values, mainly in the liquor, have been found in cerebrovascular meningitis, disseminated encephalitis, spinocerebellar degeneration, cerebral ischemia, cerebral infarction, intracerebral hematoma, subarachnoid hemorrhage, head injuries, inflammatory brain diseases, organic epilepsy, schizophrenia, and Jakob-Creutzfeld disease. (Lamerz, R., supra)

NSE has been measured on an ELECSYS analyzer using Roche product number 12133113 according to the manufacturers instructions.

CA 19-9 Carbohydrate Antigen 19-9

The CA 19-9 values measured are defined by the use of the monoclonal antibody 1116-NS-19-9. The 1116-NS-19-9-reactive determinants on a glycolipid having a molecular weight of approx. 10,000 daltons are measured. This mucin corresponds to a hapten of Lewis-a blood group determinants and is a component of a number of mucous membrane cells. (Koprowski, H., et al., Somatic Cell Genet. 5 (1979) 957-971).

3-7% of the population have the Lewis a-negative/b-negative blood group configuration and are unable to express the mucin with the reactive determinant CA 19-9. This must be taken into account when interpreting the findings.

Mucin occurs in fetal gastric, intestinal and pancreatic epithelia. Low concentrations can also be found in adult tissue in the liver, lungs, and pancreas. (Stieber, P., and Fateh-Moghadam, A., Boeringer Mannheim, Cat. No. 1536869 (engl), 1320947 (dtsch), ISBN 3-926725-07-9 dtsch/engl. Juergen Hartmann Verlag Marloffstein-Rathsberg (1993); Herlyn, M., et al., J. Clin. Immunol 2 (1982) 135-140).

CA 19-9 assay values can assist in the differential diagnosis and monitoring of patients with pancreatic carcinoma (sensitivity 70-87%) (Ritts, R. E., Jr., et al., Int. J. Cancer 33 (1984) 339-345). There is no correlation between tumor mass and the CA 19-9 assay values. However, patients with CA 19-9 serum levels above 10,000 U/mL almost always have distal metastasis.

The determination of CA 19-9 cannot be used for the early detection of pancreatic carcinoma (Steinberg, W. M., et al., Gastroenterology 90 (1986) 343-349).

In hepatobiliary carcinoma the CA 19-9 values provide a sensitivity of 50-75%. The concomitant determination of CA 724 and CEA is recommended in case of gastric carcinoma. In colorectal carcinoma, determination of CEA alone is adequate; only in rare CEA-negative cases the determination of CA 19-9 can be useful.

As the mucin is excreted exclusively via the liver, even slight cholestasis can lead to clearly elevated CA 19-9 serum levels in some cases. Elevated CA 19-9 values are also found with a number of benign and inflammatory diseases of the gastrointestinal tract and the liver, as well as in cystic fibrosis.

CA 19-9 has been measured on an ELECSYS analyzer using Roche product number 11776193 according to the manufacturers instructions.

CEA Carcinoembryonic Antigen

CEA is a monomeric glycoprotein (molecular weight approx. 180.000 dalton) with a variable carbohydrate component of approx. 45-60% (Gold, P., and Freedman, S. O., J. Exp. Med. 121 (1965) 439-462).

CEA, like AFP, belongs to the group of carcinofetal antigens that are produced during the embryonic and fetal period. The CEA gene family consists of about 17 active genes in two subgroups. The first group contains CEA and the Non-specific Cross-reacting Antigens (NCA); the second group contains the Pregnancy-Specific Glycoproteins (PSG).

CEA is mainly found in the fetal gastrointestinal tract and in fetal serum. It also occurs in slight quantities in intestinal, pancreatic, and hepatic tissue of healthy adults. The formation of CEA is repressed after birth, and accordingly serum CEA values are hardly measurable in healthy adults.

High CEA concentrations are frequently found in cases of colorectal adenocarcinoma (Stieber, P., and Fateh-Moghadam, A., supra). Slight to moderate CEA elevations (rarely >10 ng/mL) occur in 20-50% of benign diseases of the intestine, the pancreas, the liver, and the lungs (e.g. liver cirrhosis, chronic hepatitis, pancreatitis, ulcerative colitis, Crohn's Disease, emphysema) (Stieber, P., and Fateh-Moghadam, A., supra). Smokers also have elevated CEA values.

The main indication for CEA determinations is the follow-up and therapy management of colorectal carcinoma.

CEA determinations are not recommended for cancer-screening in the general population. CEA concentrations within the normal range do not exclude the possible presence of a malignant disease.

The antibodies in assay manufactured by Roche Diagnostics react with CEA and (as with almost all CEA methods) with the meconium antigen (NCA2). Cross-reactivity with NCA1 is 0.7% (Hammarstrom, S., et al., Cancer Research 49 (1989) 4852-4858, and Bormer, O. P., Tumor Biol. 12 (1991) 9-15).

CEA has been measured on an ELECSYS analyzer using Roche product number 11731629 according to the manufacturers instructions.

CYFRA 21-1

An assay for “CYFRA 21-1” specifically measures a soluble fragment of cytokeratin 19 as present in the circulation. The measurement of CYFRA 21-1 is typically based upon two monoclonal antibodies (Bodenmueller, H., et al., Int. J. Biol. Markers 9 (1994) 75-81). In the CYFRA 21-1 assay from Roche Diagnostics, Germany, the two specific monoclonal antibodies (KS 19.1 and BM 19.21) are used and a soluble fragment of cytokeratin 19 having a molecular weight of approx. 30,000 daltons is measured.

Cytokeratins are structural proteins forming the subunits of epithelial intermediary filaments. Twenty different cytokeratin polypeptides have so far been identified. Due to their specific distribution patterns they are eminently suitable for use as differentiation markers in tumor pathology. Intact cytokeratin -polypeptides are poorly soluble, but soluble fragments can be detected in serum. (Bodenmueller, H., et al., supra).

CYFRA 21-1 is a well-established marker for Non-Small-Cell Lung Carcinoma (NSCLC). The main indication for CYFRA 21-1 is monitoring the course of non-small cell lung cancer (NSCLC) (Sturgeon, C., Clinical Chemistry 48 (2002) 1151-1159).

High CYFRA 21-1 serum levels indicate an advanced tumor stage and a poor prognosis in patients with non-small-cell lung cancer (van der Gaast, A., et al., Br. J. Cancer 69 (1994) 525-528). A normal or only slightly elevated value does not rule out the presence of a tumor.

Successful therapy is documented by a rapid fall in the CYFRA 21-1 serum level into the normal range. A constant CYFRA 21-1 value or a slight or only slow decrease in the CYFRA 21-1 value indicates incomplete removal of a tumor or the presence of multiple tumors with corresponding therapeutic and prognostic consequences. Progression of the disease is often shown earlier by increasing CYFRA 21-1 values than by clinical symptomatology and imaging procedures.

It is accepted that in the primary diagnosis of pulmonary carcinoma should be made on the basis of clinical symptomatology, imaging or endoscopic procedures and intraoperative findings. An unclear circular focus in the lung together with CYFRA 21-1 values >30 ng/mL indicates with high probability the existence of primary bronchial carcinoma.

CYFRA 21-1 is also suitable for course-monitoring in myoinvasive cancer of the bladder. Good specificity is shown by CYFRA 21-1 relative to benign lung diseases (pneumonia, sarcoidosis, tuberculosis, chronic bronchitis, bronchial asthma, emphysema).

Slightly elevated values (up to 10 ng/mL) are rarely found in marked benign liver diseases and renal failure. There is no correlation with sex, age or smoking. The values for CYFRA 21-1 are also unaffected by pregnancy.

Recently it has been found that CYFRA 21-1 also is of use in detecting disease relapse and assessing treatment efficacy in the field of breast cancer (Nakata, B., et al., British J. of Cancer (2004) 1-6).

CYFRA 21-1 has been measured on an ELECSYS analyzer using Roche product number 11820966 according to the manufacturers instructions.

As mentioned further above CYFRA 21-1 is an established marker in the field of NSCLC. When developing and establishing CYFRA 21-1 for NSCLC, non-malignant disease controls derived from patients with certain lung non-malignant diseases have been used. This has been considered important to differentiate benign from malign lung diseases (H. Bodenmüiller, et al., supra).

Since only recently it is possible to detect the marker CYFRA 21-1 in a significant percentage of samples derived from patients with CRC. In addition, the presence of CYFRA 21-1 in such liquid sample obtained from an individual can be used in the assessment of colorectal cancer. Particularly in combination with other markers CYFRA 21-1 is considered to be a very useful marker in the field of CRC.

NMMT

The protein nicotinamide N-methyltransferase (NNMT; Swiss-PROT: P40261) has an apparent molecular weight of 29.6 kDa and an isoelectric point of 5.56.

NNMT catalyzes the N-methylation of nicotinamide and other pyridines. This activity is important for biotransformation of many drugs and xenobiotic compounds. The protein has been reported to be predominantly expressed in liver and is located in the cytoplasm. NNMT has been cloned from cDNA from human liver and contained a 792-nucleotide open reading frame that encoded a 264-amino acid protein with a calculated molecular mass of 29.6 kDa (Aksoy, S., et al., J. Biol. Chem. 269 (1994) 14835-14840). Little is known in the literature about a potential role of the enzyme in human cancer. In one paper, increased hepatic NNMT enzymatic activity was reported as a marker for cancer cachexia in mice (Okamura, A., et al., Jpn. J. Cancer Res. 89 (1998) 649-656). In a recent report, down-regulation of the NNMT gene in response to radiation in radiation sensitive cell lines was demonstrated (Kassem, H., et al., Int. J. Cancer 101 (2002) 454-460).

It has recently been found (WO 2004/057336) that NMMT will be of interest in the assessment of CRC. The immunoassay described in WO 2004/057336 has been used to measure the samples (CRC, healthy controls and non-malignant colon diseases) of the present study.

As the skilled artisan will appreciate there are many ways to use the measurements of two or more markers in order to improve the diagnostic question under investigation. In a quite simple, but nonetheless often effective approach, a positive result is assumed if a sample is positive for at least one of the markers investigated. This may e.g. the case when diagnosing an infectious disease, like AIDS.

Frequently, however, the combination of markers is evaluated. Preferably the values measured for markers of a marker panel, e.g. for CBP2, CYFRA 21-1 and NSE, are mathematically combined and the combined value is correlated to the underlying diagnostic question. Marker values may be combined by any appropriate state of the art mathematical method. Well-known mathematical methods for correlating a marker combination to a disease employ methods like, discriminant analysis (DA) (i.e. linear-, quadratic-, regularized-DA), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting/Bagging Methods), Generalized Linear Models (i.e. Logistic Regression), Principal Components based Methods (i.e. SIMCA), Generalized Additive Models, Fuzzy Logic based Methods, Neural Networks and Genetic Algorithms based Methods. The skilled artisan will have no problem in selecting an appropriate method to evaluate a marker combination of the present invention. Preferably the method used in correlating the marker combination of the invention e.g. to the absence or presence of CRC is selected from DA (i.e. Linear-, Quadratic-, Regularized Discriminant Analysis), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting Methods), or Generalized Linear Models (i.e. Logistic Regression). Details relating to these statistical methods are found in the following references: Ruczinski, I., et al., J. of Computational and Graphical Statistics 12 (2003) 475-511; Friedman, J. H., J. of the American Statistical Association 84 (1989) 165-175; Hastie, Trevor, Tibshirani, Robert, Friedman, Jerome, The Elements of Statistical Learning, Springer Series in Statistics, 2001; Breiman, L., Friedman, J. H., Olshen, R. A., Stone, C. J. (1984) Classification and regression trees, California: Wadsworth; Breiman, L., Random Forests, Machine Learning, 45 (2001) 5-32; Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

It is a preferred embodiment of the invention to use an optimized multivariate cut-off for the underlying combination of biological markers and to discriminate state A from state B, e.g. diseased from healthy. In this type of analysis the markers are no longer independent but form a marker panel. It could be established that combining the measurements of CBP2, NSE and CYFRA 21-1, does particularly improve the diagnostic accuracy for CRC as compared to either healthy controls or, as also assessed, as compared to healthy controls plus non-malignant disease controls. Especially the later finding is of great importance, because a patient with a non-malignant disease may require quite a different treatment as a patient with CRC.

Accuracy of a test is best described by its receiver-operating characteristics (ROC) (see especially Zweig, M. H., and Campbell, G., Clin. Chem. 39 (1993) 561-577). The ROC graph is a plot of all of the sensitivity/specificity pairs resulting from continuously varying the decision thresh-hold over the entire range of data observed.

The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example health and disease or benign versus malignant disease.

In each case, the ROC plot depicts the overlap between the two distributions by plotting the sensitivity versus 1-specificity for the complete range of decision thresholds. On the y-axis is sensitivity, or the true-positive fraction [defined as (number of true-positive test results) (number of true-positive+number of false-negative test results)]. This has also been referred to as positivity in the presence of a disease or condition. It is calculated solely from the affected subgroup. On the x-axis is the false-positive fraction, or 1- specificity [defined as (number of false-positive results)/ (number of true-negative+number of false-positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/1-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. (If the ROC plot falls completely below the 45° diagonal, this is easily remedied by reversing the criterion for “positivity” from “greater than” to “less than” or vice versa.) Qualitatively, the closer the plot is to the upper left corner, the higher the overall accuracy of the test.

One convenient goal to quantify the diagnostic accuracy of a laboratory test is to express its performance by a single number. The most common global measure is the area under the ROC plot. By convention, this area is always >0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area =1.0).

Combining measurements of CBP2 with other recently discovered markers, like CYFRA 21-1 or NMMT or with known markers like CEA and NSE, or with other markers of CRC yet to be discovered, leads and will lead, respectively, to further improvements in assessment of CRC.

The following examples, references, and sequence listing are provided to aid the understanding of the present invention, the true scope of which is set forth in the appended claims. It is understood that modifications can be made in the procedures set forth without departing from the spirit of the invention.

Abbreviations

ABTS 2,2′-azino-di-[3-ethylbenzthiazoline sulfonate (6)] diammonium salt
BSA bovine serum albumin
cDNA complementary DNA
CHAPS (3-[(3-cholamidopropyl)-dimethylammonio]-1-propane-sulfonate)
DMSO dimethyl sulfoxide
DTT dithiothreitol
EDTA ethylene diamine tetraacetic acid
ELISA enzyme-linked immunosorbent assay
HRP horseradish peroxidase
IAA iodacetamid
IgG immunoglobulin G
IEF isoelectric focusing
IPG immobilized pH gradient
LDS lithium dodecyl sulfate
MALDI-TOF matrix-assisted laser desorption/ionization-time of flight mass spectrometry
MES mesity, 2,4,6-trimethylphenyl
OD optical density
PAGE polyacrylamide gel electrophoresis
PBS phosphate buffered saline
PI isoelectric point
RTS rapid translation system
SDS sodium dodecyl sulfate

### SPECIFIC EMBODIMENTS

### Example 1

### Identification of CBP2 as a Potential Colorectal Cancer Marker

Sources of Tissue

In order to identify tumor-specific proteins as potential diagnostic markers for colorectal cancer, analysis of three different kinds of tissue using proteomics methods is performed.

In total, tissue specimen from 10 patients suffering from colorectal cancer are analyzed. From each patient three different tissue types are collected from therapeutic resections: tumor tissue (>80% tumor) (T), adjacent healthy tissue (N) and stripped mucosa from adjacent healthy mucosa (M). The latter two tissue types serve as matched healthy control samples. Tissues are immediately snap frozen after resection and stored at −80° C. before processing. Tumors are diagnosed by histopathological criteria.

Tissue Preparation

0.8-1.2 g of frozen tissue are put into a mortar and completely frozen by liquid nitrogen. The tissue is pulverized in the mortar, dissolved in the 10-fold volume (w/v) of lysis buffer (40 mM Na-citrate, 5 mM MgCl₂, 1% Genapol X-080, 0.02% Na-azide, Complete® EDTA-free [Roche Diagnostics GmbH, Mannheim, Germany, Cat. No. 1 873 580]) and subsequently homogenized in a Wheaton® glass homogenizer (20×loose fitting, 20×tight fitting). 3 ml of the homogenate are subjected to a sucrose-density centrifugation (10-60% sucrose) for 1 h at 4,500×g. After this centrifugation step three fractions are obtained. The fraction on top of the gradient contains the soluble proteins and is used for further analysis.

Sample Preparation for LC-ESI-MSMS-Analysis

The protein concentration of the soluble protein fraction is determined using Bio-Rad® protein assay (Cat.No. 500-0006; Bio-Rad Laboratories GmbH, München, Germany) following the instructions of the supplier's manual. To a volume corresponding to 200 μg of protein 4 ml reduction buffer (9 M urea, 2 mM DTT, 100 mM KH₂PO₄, pH 8.2 NaOH) is added and incubated for 1 h. The solution is concentrated to 250 μl in an AMICON Ultra 10 kD device (Millipore GmbH, Schwalbach, Germany). For alkylation the 250 μl are transferred into 1 ml alkylation buffer (9 M urea, 4 mM iodoacetamide, 100 mM KH₂PO₄, pH 8.2 NaOH), incubated for 6 h and subsequently concentrated in an AMICON Ultra 10 kD device to 250 μl. For washing 1 ml 9 M urea is added and again concentrated in an AMICON Ultra 10 kD device to 250 μl. Washing is repeated three-times.

For protease digestion the concentrated solution is diluted to 2.5 M urea and incubated with 4 μg trypsin (Proteomics grade, Roche Diagnostics GmbH, Mannheim, Germany) over night. The digestion is stopped by adding 1 ml 1% formic acid and analyzed.

LC-ESI-MSMS-Analysis

The tryptic digest (500 μl) is separated on a two-dimensional Nano-HPLC-System (Ultimate, Famos, Switchos; LC Packings, Idstein, Germany) consisting of a SCX and a RP Pepmep C18 column (LC Packings, Idstein, Germany). The 11 SCX fractions (step elution with 0, 5, 10, 25, 50, 100, 200, 300, 400, 500, 1,500 mM NH₄Ac) where successively further separated on the RP column with a 90 min gradient (5-95% acetonitrile) and online analyzed using data dependent scans with an ESI-MS ion trap (LCQ deca XP; Thermo Electron, Massachusetts, USA; see Table 2 for parameters). For each sample three runs are performed. The raw data are processed with Bioworks 3.1 software (Thermo Electron, Massachusetts, USA) using the parameters listed in Table 2. The resulting lists of identified peptides and proteins from replicate runs where combined.

The protein CBP2 is identified with the sequences given in Table 1.

Detection of CBP2 as a Potential Marker for Colorectal Cancer

For each patient the identified proteins and the number of corresponding peptides from the tumor sample are compared to the accordant results from adjacent normal tissue and from stripped normal mucosa tissue. By this means, protein CBP2 is found to be specifically expressed or strongly overexpressed in tumor tissue and not or less detectable or less strongly expressed in healthy control tissue. It therefore—amongst many other proteins—qualifies as a candidate marker for use in the diagnosis of colorectal cancer.

The protein CBP2 was strongly over-represented in tumor tissue from patients suffering from colorectal cancer. The following peptide sequences of the protein CBP2 were identified with Bioworks 3.1 form LCQ-MS²-data in tumor tissue:

TABLE 1

| | | |
|---|---|---|
| i | LCSRLGPSSVSFADDFVR | (SEQ ID NO:3) |
| ii | HLAGLGLTEAIDKNK | (SEQ ID NO:4) |
| iii | LFYADHPFIFLVR | (SEQ ID NO:5) |
| iv | LSSLIILMPHHVEPLER | (SEQ ID NO:6) |
| v | SALQSINEWAAQTTDGKLPEVTK | (SEQ ID NO:7) |

TABLE 2
MSMS-data acquisition and Bioworks 3.1 search parameters

| | | |
|---|---|---|
| MSMS-data | MS exclusion | 350-2,000 Da for |
| acquisition | | precursor ions |
| | Repeat count | 2 |
| | Repeat duration | 0.25 min |
| | Exclusion list size | 25 |
| | Exclusion duration | 5 min |
| | Exclusion mass width | low 0.5 Da, high 1.5 Da |
| Bioworks | Number of ions | 35 |
| | Minimal ion intensity | 100,000 counts |
| | Precursor mass | 1.2 Da |
| | tolerance | |
| | Fragment mass | 1.4 Da |
| | tolerance | |
| | Xcorr | >2; 2.5; 3 (z = 1; 2; 3) |
| | dCn | >0.1 |
| | Sp | >500 |
| Databases | | Swissprot; Humangp |
| | | (assembled by Roche |
| | | Bioinformatics) |

### Example 2

### Generation of Antibodies to the Colorectal Cancer Marker Protein CBP2

Polyclonal antibody to the colorectal cancer marker protein CBP2 is generated for further use of the antibody in the measurement of serum and plasma and blood levels of CBP2 by immunodetection assays, e.g. Western Blotting and ELISA.

Recombinant Protein Expression in E. coli

In order to generate antibodies to CBP2, recombinant expression of the protein is performed for obtaining immunogens. The expression is done applying a combination of the RTS 100 expression system and E. coli. In a first step, the DNA sequence is analyzed and recommendations for high yield cDNA silent mutational variants and respective PCR-primer sequences are obtained using the “ProteoExpert RTS E. coli HY” system. This is a commercial web based service (www.proteoexpert.com). Using the recommended primer pairs, the “RTS 100 E. coli Linear Template Generation Set, His-tag” (Roche Diagnostics GmbH, Mannheim, Germany, Cat.No. 3186237) system to generate linear PCR templates from the cDNA and for in vitro transcription and expression of the nucleotide sequence coding for the CBP2 protein is used. For Western-blot detection and later purification, the expressed protein contains a His-tag. The best expressing variant is identified. All steps from PCR to expression and detection are carried out according to the instructions of the manufacturer. The respective PCR product, containing all necessary T7 regulatory regions (promoter, ribosomal binding site and T7 terminator) is cloned into the pBAD TOPO® vector (Invitrogen, Karlsruhe, Germany, Cat. No. K 4300/01) following the manufacturer's instructions. For expression using the T7 regulatory sequences, the construct is transformed into E. coli BL 21 (DE 3) (Studier, F. W., et al., Methods Enzymol. 185 (1990) 60-89) and the transformed bacteria are cultivated in a 11 batch for protein expression.

Purification of His-CBP2 fusion protein is done following standard procedures on a Ni-chelate column. Briefly, 11 of bacteria culture containing the expression vector for the His-CBP2 fusion protein is pelleted by centrifugation. The cell pellet is resuspended in lysis buffer, containing phosphate, pH 8.0, 7 M guanidium chloride, imidazole and thioglycerole, followed by homogenization using a ULTRA-TURRAX. Insoluble material is pelleted by high speed centrifugation and the supernatant is applied to a Ni-chelate chromatographic column. The column is washed with several bed volumes of lysis buffer followed by washes with buffer, containing phosphate, pH 8,0 and urea. Finally, bound antigen is eluted using a phosphate buffer containing SDS under acid conditions.

Production of Monoclonal Antibodies Against the CBP2

a) Immunization of Mice

12 week old A/J mice are initially immunized intraperitoneally with 100 μg CBP2. This is followed after 6 weeks by two further intraperitoneal immunizations at monthly intervals. In this process each mouse is administered 100 μg CBP2 adsorbed to aluminum hydroxide and 10⁹ germs of Bordetella pertussis. Subsequently the last two immunizations are carried out intravenously on the 3rd and 2nd day before fusion using 100 μg CBP2 in PBS buffer for each.

b) Fusion and Cloning

Spleen cells of the mice immunized according to a) are fused with myeloma cells according to Galfre, G., and Milstein, C., Methods in Enzymology 73 (1981) 3-46. In this process ca. 1*10⁸ spleen cells of the immunized mouse are mixed with 2×10⁷ myeloma cells (P3X63-Ag8-653, ATCC CRL1580) and centrifuged (10 min at 300×g and 4° C.). The cells are then washed once with RPMI 1640 medium without fetal calf serum (FCS) and centrifuged again at 400×g in a 50 ml conical tube. The supernatant is discarded, the cell sediment is gently loosened by tapping, 1 ml PEG (molecular weight 4,000, Merck, Darmstadt) is added and mixed by pipetting. After 1 min in a water-bath at 37° C., 5 ml RPMI 1640 without FCS is added drop-wise at room temperature within a period of 4-5 min. Afterwards 5 ml RPMI 1640 containing 10% FCS is added drop-wise within ca. 1 min, mixed thoroughly, filled to 50 ml with medium (RPMI 1640+10% FCS) and subsequently centrifuged for 10 min at 400×g and 4° C. The sedimented cells are taken up in RPMI 1640 medium containing 10% FCS and sown in hypoxanthine-azaserine selection medium (100 mmol/l hypoxanthine, 1 μg/ml azaserine in RPMI 1640+10% FCS). Interleukin 6 at 100 U/ml is added to the medium as a growth factor.

After ca. 10 days the primary cultures are tested for specific antibody. CBP2-positive primary cultures are cloned in 96-well cell culture plates by means of a fluorescence activated cell sorter. In this process again interleukin 6 at 100 U/ml is added to the medium as a growth additive.

c) Immunoglobulin Isolation from the Cell Culture Supernatants

The hybridoma cells obtained are sown at a density of 1×10⁵ cells per ml in RPMI 1640 medium containing 10% FCS and proliferated for 7 days in a fermenter (Thermodux Co., Wertheim/Main, Model MCS-104XL, Order No. 144-050). On average concentrations of 100 μg monoclonal antibody per ml are obtained in the culture supernatant. Purification of this antibody from the culture supernatant is carried out by conventional methods in protein chemistry (e.g. according to Bruck, C., et al., Methods Enzymol. 121 (1986) 587-695).

Generation of Polyclonal Antibodies

a) Immunization

For immunization, a fresh emulsion of the protein solution (100 μg/ml protein CBP2) and complete Freund's adjuvant at the ratio of 1:1 is prepared. Each rabbit is immunized with 1 ml of the emulsion at days 1, 7, 14 and 30, 60 and 90. Blood is drawn and resulting anti-CBP2 serum used for further experiments as described in examples 3 and 4.

b) Purification of IgG (Immunoglobulin G) from Rabbit Serum by Sequential Precipitation with Caprylic Acid and Ammonium Sulfate

One volume of rabbit serum is diluted with 4 volumes of acetate buffer (60 mM, pH 4.0). The pH is adjusted to 4.5 with 2 M Tris-base. Caprylic acid (25 μl/ml of diluted sample) is added drop-wise under vigorous stirring. After 30 min the sample is centrifuged (13,000×g, 30 min, 4° C.), the pellet discarded and the supernatant collected. The pH of the supernatant is adjusted to 7.5 by the addition of 2 M Tris-base and filtered (0.2 μm).

The immunoglobulin in the supernatant is precipitated under vigorous stirring by the drop-wise addition of a 4 M ammonium sulfate solution to a final concentration of 2 M. The precipitated immunoglobulins are collected by centrifugation (8,000×g, 15 min, 4° C.).

The supernatant is discarded. The pellet is dissolved in 10 mM NaH₂PO₄/NaOH, pH 7.5, 30 mM NaCl and exhaustively dialyzed. The dialysate is centrifuged (13,000×g, 15 min, 4° C.) and filtered (0.2 μm).

Biotinylation of Polyclonal Rabbit IgG

Polyclonal rabbit IgG is brought to 10 mg/ml in 10 mM NaH₂PO₄/NaOH, pH 7.5, 30 mM NaCl. Per ml IgG solution 50 μl Biotin-N-hydroxysuccinimide (3.6 mg/ml in DMSO) are added. After 30 min at room temperature, the sample is chromatographed on Superdex 200 (10 mM NaH₂PO₄/NaOH, pH 7.5, 30 mM NaCl). The fraction containing biotinylated IgG are collected. Monoclonal antibodies are biotinylated according to the same procedure.

Digoxigenylation of Polyclonal Rabbit IgG

Polyclonal rabbit IgG is brought to 10 mg/ml in 10 mM NaH₂PO₄/NaOH, 30 mM NaCl, pH 7.5. Per ml IgG solution 50 μl digoxigenin-3-O-methylcarbonyl-ε-aminocaproic acid-N-hydroxysuccinimide ester (Roche Diagnostics, Mannheim, Germany, Cat. No. 1 333 054) (3.8 mg/ml in DMSO) are added. After 30 min at room temperature, the sample is chromatographed on SUPERDEX 200 (10 mM NaH₂PO₄/NaOH, pH 7.5, 30 mM NaCl). The fractions containing digoxigenylated IgG are collected. Monoclonal antibodies are labeled with digoxigenin according to the same procedure.

### Example 3

### Western Blotting for the Detection of CBP2 in Human Colorectal Cancer Tissue Using Polyclonal Antibody as Generated in Example 2

Tissue lysates from tumor samples and healthy control samples are prepared as described in Example 1, “Tissue preparation”.

SDS-PAGE and Western-Blotting are carried out using reagents and equipment of Invitrogen, Karlsruhe, Germany. For each tissue sample tested, 10 μg of tissue lysate are diluted in reducing NuPAGE (Invitrogen) SDS sample buffer and heated for 10 min at 95° C. Samples are run on 4-12% NuPAGE gels (Tris-Glycine) in the MES running buffer system. The gel-separated protein mixture is blotted onto nitrocellulose membranes using the Invitrogen XCell II Blot Module (Invitrogen) and the NuPAGE transfer buffer system. The membranes are washed 3 times in PBS/0.05% TWEEN 20 and blocked with Roti-Block blocking buffer (A151.1; Carl Roth GmbH, Karlsruhe, Germany) for 2 h. The primary antibody, polyclonal rabbit anti-CBP2 serum (generation described in Example 2), is diluted 1: 10,000 in Roti-Block blocking buffer and incubated with the membrane for 1 h. The membranes are washed 6 times in PBS/0.05% TWEEN 20. The specifically bound primary rabbit antibody is labeled with an POD-conjugated polyclonal sheep anti-rabbit IgG antibody, diluted to 10 mU/ml in 0.5×Roti-Block blocking buffer. After incubation for 1 h, the membranes are washed 6 times in PBS/0.05% TWEEN 20. For detection of the bound POD-conjugated anti-rabbit antibody, the membrane is incubated with the Lumi-Light^{PLUs} Western Blotting Substrate (Order-No. 2015196, Roche Diagnostics GmbH, Mannheim, Germany) and exposed to an autoradiographic film.

### Example 4

### ELISA for the Measurement of CBP2 in Human Serum and Plasma Samples

For detection of CBP2 in human serum or plasma, a sandwich ELISA is developed. For capture and detection of the antigen, aliquots of the anti-CBP2 polyclonal antibody (see Example 2) are conjugated with biotin and digoxigenin, respectively.

Streptavidin-coated 96-well microwell plates are incubated with 100 μl biotinylated anti-CBP2 polyclonal antibody for 60 min at 10 μg/ml in 10 mM phosphate, pH 7.4, 1% BSA, 0.9% NaCl and 0.1% TWEEN 20. After incubation, plates are washed three times with 0.9% NaCl , 0.1% TWEEN 20. Wells are then incubated for 2 h with either a serial dilution of the recombinant protein (see Example 2) as standard antigen or with diluted liquid samples obtained from patients. After binding of CBP2, plates are washed three times with 0.9% NaCl, 0.1% TWEEN 20. For specific detection of bound CBP2, wells are incubated with 100 μl of digoxigenylated anti-CBP2 polyclonal antibody for 60 min at 10 μg/ml in 10 mM phosphate, pH 7.4, 1% BSA, 0.9% NaCl and 0.1% TWEEN 20. Thereafter, plates are washed three times to remove unbound antibody. In a next step, wells are incubated with 20 mU/ml anti-digoxigenin-POD conjugates (Roche Diagnostics GmbH, Mannheim, Germany, Catalog No. 1633716) for 60 min in 10 mM phosphate, pH 7.4, 1% BSA, 0.9% NaCl and 0.1% TWEEN 20. Plates are subsequently washed three times with the same buffer. For detection of antigen-antibody complexes, wells are incubated with 100 μl ABTS solution (Roche Diagnostics GmbH, Mannheim, Germany, Catalog No. 11685767) and OD is measured after 30-60 min at 405 nm with an ELISA reader.

### Example 5

### ROC Analysis to Assess Clinical Utility in Terms of Diagnostic Accuracy

Accuracy is assessed by analyzing individual liquid samples obtained from well-characterized patient cohorts, i.e., 50 patients having undergone colonoscopy and found to be free of adenoma or CRC, 50 patients diagnosed and staged as Tᵢₛ-3, N0, M0 of CRC, and 50 patients diagnosed with progressed CRC, having at least tumor infiltration in at least one proximal lymph node or more severe forms of metastasis, respectively. CEA as measured by a commercially available assay (Roche Diagnostics, CEA-assay (Cat. No. 1 173 1629 for ELECSYS systems immunoassay analyzer) and CBP2 measured as described above are quantified in a serum obtained from each of these individuals. ROC-analysis is performed according to Zweig, M. H., and Campbell, supra. Discriminatory power for differentiating patients in the group Tᵢₛ-3, N0, M0 from healthy individuals for the combination of CBP2 with the established marker CEA is calculated by regularized discriminant analysis (Friedman, J. H., Regularized Discriminant Analysis, Journal of the American Statistical Association 84 (1989) 165-175).

Preliminary data indicate that CBP2 may also be very helpful in the follow-up of patients after surgery.

What is claimed is:

1. A method for assessing colorectal cancer in a patient comprising:
measuring in a sample from said patient a concentration of CBP2 (collagen-binding protein 2), and
using the concentration measured in the assessment of colorectal cancer.

2. The method of claim 1 wherein said sample is serum.

3. The method of claim 1 wherein said sample is plasma.

4. The method of claim 1 wherein said sample is whole blood.

5. The method of claim 1 further comprising the step of measuring in said sample a concentration of a known marker of colorectal cancer and including the concentration of the known marker in the assessment of colorectal cancer.

6. The method of claim 5 wherein said known marker is selected from the group consisting of neuron-specific enolase (NSE), CYFRA 21-1, nicotinamide N-methyltransferase (NNMT), carbohydrate antigen 19-9 (CA 19-9), CA 72-4, and carcinoembryonic antigen (CEA).

7. A marker panel comprising a specific binding agent for CBP2 and a specific binding agent for a known marker of colorectal cancer.

8. The marker panel of claim 7 wherein said known marker is selected from the group consisting of neuron-specific enolase (NSE), CYFRA 21-1, nicotinamide N-methyltransferase (NNMT), carbohydrate antigen 19-9 (CA 19-9), CA 72-4, and carcinoembryonic antigen (CEA).

9. A kit for assessing colorectal cancer in a patient, said kit comprising reagents for measuring CBP2.

10. The kit of claim 9 further comprising reagents for measuring a known marker of colorectal cancer, wherein said known marker is selected from the group consisting of neuron-specific enolase (NSE), CYFRA 21-1, nicotinamide N-methyltransferase (NNMT), carbohydrate antigen 19-9 (CA 19-9), CA 72-4, and carcinoembryonic antigen (CEA).
